(19) United States
(12) Patent Application Publication
(10) Pub. No.: US 2018/0333211 A1
(43) Pub. Date: Nov. 22, 2018

(54) System for Endovaginal Delivery of Therapeutic Energies

(71) Applicant: Viveve, Inc., Englewood, CO (US)

(72) Inventors: Perry J. Tomasetti, Pepperell, MA (US); James Atkinson, Greenwood Village, CO (US); David Black, Cameron, MT (US)

(73) Assignee: Viveve, Inc., Englewood, CO (US)

(21) Appl. No.: 15/983,958

(22) Filed: May 18, 2018

Related U.S. Application Data
(60) Provisional application No. 62/580,148, filed on Nov. 1, 2017.
(60) Provisional application No. 62/508,856, filed on May 19, 2017.

(51) Int. Cl. A61B 34/20; A61B 8/08; A61N 2/02; A61N 2/00; A61N 5/04; A61N 7/00

(52) U.S. Cl. CPC A61B 34/20; A61B 8/0841; A61N 2/02; A61N 1/40; A61N 5/045; A61N 7/00; A61N 2/002

(57) ABSTRACT

A system and method for endovaginal delivery of therapeutic treatment energies. The system and method include an applicator having a body with a treatment window. The treatment window may include one or more transducers and one or more electrodes for delivering the therapeutic treatment energies to a target tissue in the vagina. The system and method may include a rollerball tip, an optical window, and ultrasonic imaging. The vagina may be mapped and the applicator may be located at a target tissue selected based on the map to deliver one or more treatment energies.

### CROSS-REFERENCE TO RELATED APPLICATIONS

This application claims priority to U.S. Provisional Patent Application No. 62/508,856, filed May 19, 2017, and U.S. Provisional Patent Application No. 62/580,148, filed Nov. 1, 2017, both of which are hereby incorporated by reference in their entireties.

### TECHNICAL FIELD OF THE INVENTION

The present invention relates generally to systems and methods for endovaginal delivery of therapeutic energies.

### BACKGROUND OF THE INVENTION

There is a need to treat Stress Urinary Incontinence (SUI) in women and men. Stress Urinary Incontinence is the inability to prevent urine flow during physical activity (e.g., running, exercise, sport and leisure activities, etc.) or transient events (e.g., coughing, sneezing, bending, lifting, etc.) that increase abdominal pressures above a baseline. Stress Urinary Incontinence may affect a significantly large number of post-partum women and other females, particular in older age. Men may also suffer from Stress Urinary Incontinence. One treatment for Stress Urinary Incontinence is surgical intervention. However, this may be costly and invasive. Surgical interventions may also result in complications. Another treatment for Stress Urinary Incontinence is a laser based treatment. However, laser based treatments are only nominally effective and may only be effective for a short time. Such laser based treatments are not durable. There exists a need for improved devices, systems, and methods for treatment of Stress Urinary Incontinence that are both effective and durable.

### BRIEF SUMMARY OF THE INVENTION

According to an embodiment of the invention, a system for delivery of therapeutic energies to a target tissue includes an applicator having a body; a treatment window located on the body of the applicator; and a moveable member coupled to the applicator, the moveable member configured to move with respect to the body, wherein the moveable member is configured to move the treatment window into alignment with the target tissue for delivery of a treatment energy.

In an embodiment of the invention, the moveable member is a rollerball tip coupled to a distal end of the body. In an embodiment of the invention, the rollerball tip includes an encoder configured to sense a rotational and longitudinal position of the applicator. In an embodiment of the invention, the treatment window is configured to deliver the treatment energy automatically based on the sensed position. In an embodiment of the invention, the treatment window is configured to delivery repeated pulses automatically based on a preselected rotational or longitudinal position sensed by the encoder. In an embodiment of the invention, the rollerball tip rotates relative to the body.

In an embodiment of the invention, the moveable member is a motorized portion coupling the treatment window to the applicator. In an embodiment of the invention, the motorized portion is configured to move the treatment window rotational and longitudinally along the body of the applicator. In an embodiment of the invention, the motorized portion is configured to align the treatment window with the target tissue for delivery of the treatment energy.

In an embodiment of the invention, the applicator is configured to scout an area to determine one or more target tissues, and the moveable member moves the treatment window from a zero position to a treatment position aligned with one of the one or more target tissues to deliver the treatment energy to the one of the one or more target tissues. In an embodiment of the invention, the moveable member moves the treatment window from the treatment position to the zero position after delivery of the treatment energy. In an embodiment of the invention, wherein the moveable member moves the treatment window automatically.

In an embodiment of the invention, a system for endovaginal delivery of therapeutic energies, the system including: an applicator including: a body; and a treatment window located on a side of the body, the treatment window including one or more transducers, wherein the applicator is configured to be positioned within a vagina of a patient and, wherein the treatment window is configured to be aligned with a target tissue in the vagina and the one or more transducers are configured to deliver a treatment energy to the target tissue.

In an embodiment of the invention, the system includes an activation device configured to turn the one or more transducers on and off. In an embodiment of the invention, the treatment energy is one or more of Therapeutic Ultrasound (TUS), Monopolar Radiofrequency (MRF), Microwave Radiofrequency (MWRF), Alternating Magnetic Field (AMF), or combinations thereof.

In an embodiment of the invention, the body of the applicator includes a substantially cylindrical portion and a rounded distal tip and wherein the treatment window is located on the substantially cylindrical portion. In an embodiment of the invention, the treatment window creates a substantially flat portion on the substantially cylindrical portion of the body. In an embodiment of the invention, the treatment window is moveable longitudinally along the body and rotationally around the body and wherein the treatment window is configured to map an area of the vagina for selecting one or more target tissues.

In an embodiment of the invention, the one or more transducers form a transducer array. In an embodiment of the invention, the transducer array is a therapeutic ultrasound array or linear transducer array. In an embodiment of the invention, the system further includes one or more electrodes. In an embodiment of the invention, the one or more electrodes form an electrode array. In an embodiment of the invention, the one or more transducers and the one or more electrodes deliver one or more treatment energies including Therapeutic Ultrasound (TUS), Monopolar Radiofrequency (MRF), Microwave Radiofrequency (MWRF), Alternating Magnetic Field (AMF), or combinations thereof. In an embodiment of the invention, the system further includes one or more of ultrasound imaging, a rollerball tip, or an optical window on the applicator. In an embodiment of the invention, the rollerball tip and optical window include an encoder for sensing rotational and longitudinal position changes of the applicator. In an embodiment of the invention, the applicator is located with ultrasound imaging and the treatment window delivers a therapeutic ultrasound treatment energy.

In an embodiment of the invention, a method for treating a target tissue with therapeutic energies includes inserting an applicator with a treatment window into a vagina of a patient; locating the treatment window adjacent a target tissue of the vagina; activating one or more transducers in the treatment window; delivering a treatment energy to the target tissue with the one or more transducers; relocating the applicator to a second target tissue in the vagina without removing the applicator from the vagina; and delivering the treatment energy to the second target tissue.

In an embodiment of the invention, the method includes locating the applicator within the vagina with one or more of direct observation, inference relative to anatomical landmarks on an anterior vulva, or ultrasonic imaging. In an embodiment of the invention, direct observation includes observation of the length and location of the urethral prominence. In an embodiment of the invention, the target tissue is one or more of mucosa, lamina propria, or fibromuscular layers.

In an embodiment of the invention, the method includes sensing rotational and longitudinal position changes of the applicator and automatically activating the one or more transducers based on the sensed position changes. In an embodiment of the invention, the method includes activating one or more transducers includes operating an activation device.

In an embodiment of the invention, the method includes automatically pulsing treatment energies when the applicator is located at at least one of the target tissue or the second target tissue. In an embodiment of the invention, the method includes mapping an area of the vagina by moving the treatment window longitudinally along and rotational around the applicator; and selecting one or more target tissues based on the area mapped.

Additional features, advantages, and embodiments of the invention are set forth or apparent from consideration of the following detailed description and drawings. Moreover, it is to be understood that both the foregoing summary of the invention and the following detailed description are exemplary and intended to provide further explanation without limiting the scope of the invention as claimed.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A shows a side view of an applicator, according to an embodiment of the present invention.

FIG. 1B shows an end view of the applicator of FIG. 1A.

FIG. 1C shows a top view of the applicator of FIG. 1A.

FIG. 1D shows an end view of the applicator of FIG. 1A, where the applicator is rotated 90° about the longitudinal axis of the applicator relative to the end view in FIG. 1B.

FIG. 2A shows a top view of an applicator with a rollerball encoder tip, according to another embodiment of the present invention.

FIG. 2B shows an end view of the applicator of FIG. 2A.

FIG. 2C shows a side view of the applicator of FIG. 2A.

FIG. 3 shows a top view of an applicator with an optical motion encoder window, according to another embodiment of the present invention.

FIG. 4 shows a top view of an applicator with rotational and longitudinal motion sensing, according to another embodiment of the present invention.

FIG. 5 shows a top view of an applicator with a transducer array capable of automated rotation and translation, according to another embodiment of the present invention.

### DETAILED DESCRIPTION OF THE INVENTION

To address the need for improved devices, systems, and methods for treatment of Stress Urinary Incontinence that are both effective and durable, embodiments of the current invention provide endovaginal delivery of therapeutic energies to effect tissue remodeling in the mucosa, lamina propria and/or fibromuscular layers of a patient. The treatment for Stress Urinary Incontinence or other conditions may include a workstation with a device console. The device console may include one or more of a power supply, display, control electronics, and one or more delivery systems. The workstation and device console may provide visualization of the target tissue. The workstation may allow for selecting, scaling, and activating treatment parameters for the delivery of the treatment. The treatments may be treatment energies and may be delivered individually or in combination with other treatment energies. Non-limiting examples of treatment energies are Therapeutic Ultrasound (TUS), Monopolar Radiofrequency (MRF), Microwave Radiofrequency (MWRF), or Alternating Magnetic Field (AMF). Other suitable treatment energies may be provided in the treatment.

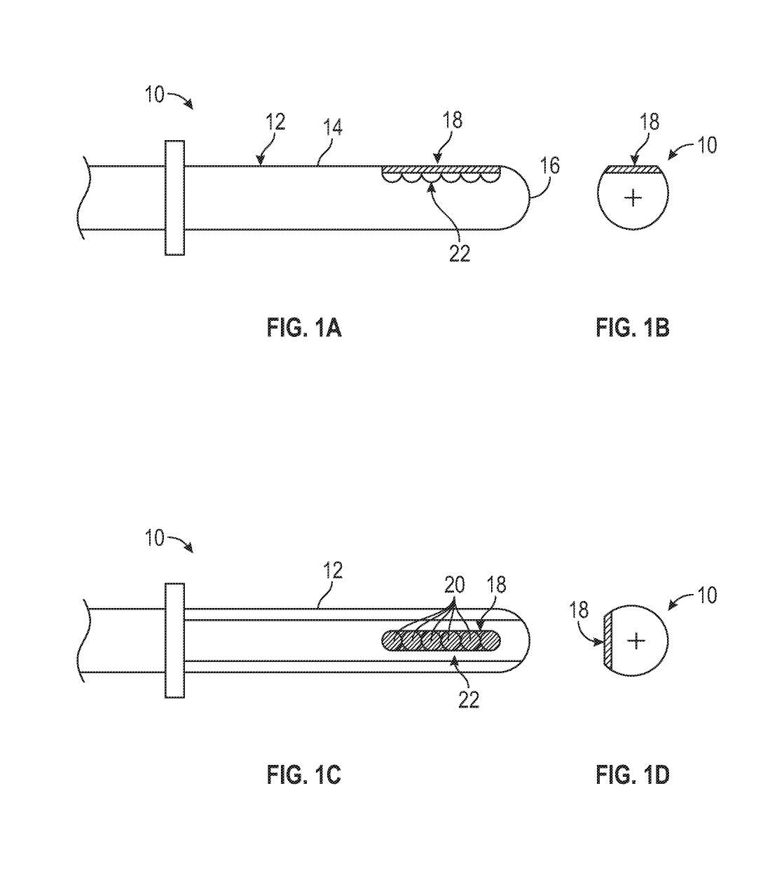

The workstation may include an applicator for performing the treatment. An exemplary applicator 10 according to one embodiment is shown in FIGS. 1A-1D. The applicator 10 may have a body 12. The body 12 may have a substantially cylindrical portion 14 and a rounded tip 16. The rounded tip 16 may be the distal tip and/or at a distal end of the body 12 and/or the applicator 10. The applicator 10 and/or body 12 may be sized and shaped for entry into a patient's vagina. The applicator 10 may have a treatment window 18 on the substantially cylindrical portion 14 of the body 12. However, other placements of the treatment window 18 are contemplated. The treatment window 18 may include one or more transducers 20 for delivering the treatment energy or energies. The one or more transducers 20 may be a transducer array 22, such as a Therapeutic Ultrasound (TUS) array. The transducer array 22 may be a linear transducer array. The TUS or transducer array 22 may be side facing and located on one side of the applicator 10. The treatment window 18 may be located on an exterior side surface of the cylindrical portion 14 of the body 12. As may be seen from FIGS. 1B and 1D, the location of the treatment window, including the one or more transducers 20 and/or transducer array 22, on the side of the applicator 10 may result in a substantially horizontal or flat portion of the body 12.

The applicator 10 may be placed in the vagina and the treatment window 18 may be placed on a first target tissue. The treatment window 18 may be aligned facing the first target tissue. The applicator 10 and treatment window 18 may be located at the first target tissue by direct observation and/or may be inferred relative to anatomical landmarks on the anterior vulva. Direct observation may be the observation of the length and/or location of the urethral prominence. The anatomical landmarks may be the location of the visible portion of the clitoris and/or the location of the urethral papilla, however other anatomical landmarks are contemplated. For example, the applicator 10 may be navigated in the vagina until the direct observation and/or inferred positioning determines the applicator 10 and treatment window 18 are aligned with the first target tissue.

Once located, the operator of the workstation may activate the one or more transducers 20 and/or transducer array 22 to deliver treatment energy to the target tissue. The treatment energy may be one or more of Therapeutic Ultrasound (TUS), Monopolar Radiofrequency (MRF), Microwave Radiofrequency (MWRF), or Alternating Magnetic Field (AMF). The activation may be provided by a foot activation device or a hand activation device. The activation device may be a switch, button, trigger, or other device for activating a transducer. The operator may provide the activation through the closure and release of the activation device.

After delivery of the treatment energy (such as the TUS energy), the applicator 10 may be moved to a second or subsequent location within the vagina for treatment of a second or subsequent target tissue. The movement of the applicator 10 to the second location may be through direct observation or be inferred relative to anatomical landmarks, as previously described. The operator may activate the one or more transducers 20 and/or the transducer array 22 a second or subsequent time and deliver treatment energy to the second target tissue. After delivery of the second or subsequent treatment energy, the applicator 10 may be moved in any of the previously described manners to a third or subsequent target tissue location. The process may be repeated, as needed, at any number of target tissue locations until the full treatment is provided. Alternatively, the process may be completed after the treatment at the first target tissue and the applicator 10 may be removed from the vagina. After the delivery of the final treatment energy, after all relocations and applications to target tissues has been performed, the applicator 10 may be removed from the vagina.

With continued reference to FIGS. 1A-1D, the applicator 10 may also include Ultrasound Imaging (USI). The USI (not depicted) may be provided in the treatment window 18 with the transducer array 22, such as the TUS array. In operation, the treatment window 18 may be placed on the target tissue using ultrasonic imaging of anatomical landmarks. That is, instead of direct or inferred observation or in addition to direct or inferred observation, USI may locate the applicator 10 at the target tissue. In contrast to location by inference relative to anatomical landmarks, ultrasonically imaging (e.g. with USI) the intended treatment area may improve the accuracy of locating the applicator 10 at the target tissue. This may improve the accuracy of energy deposition and may help include or avoid certain tissues that may respond, or be sensitive to the treatment energy. After placement, activation by the operator may be performed as previously described. When the applicator 10 is moved within the vagina to additional target tissues, the USI may assist in the placement using ultrasonic imaging of anatomical landmarks.

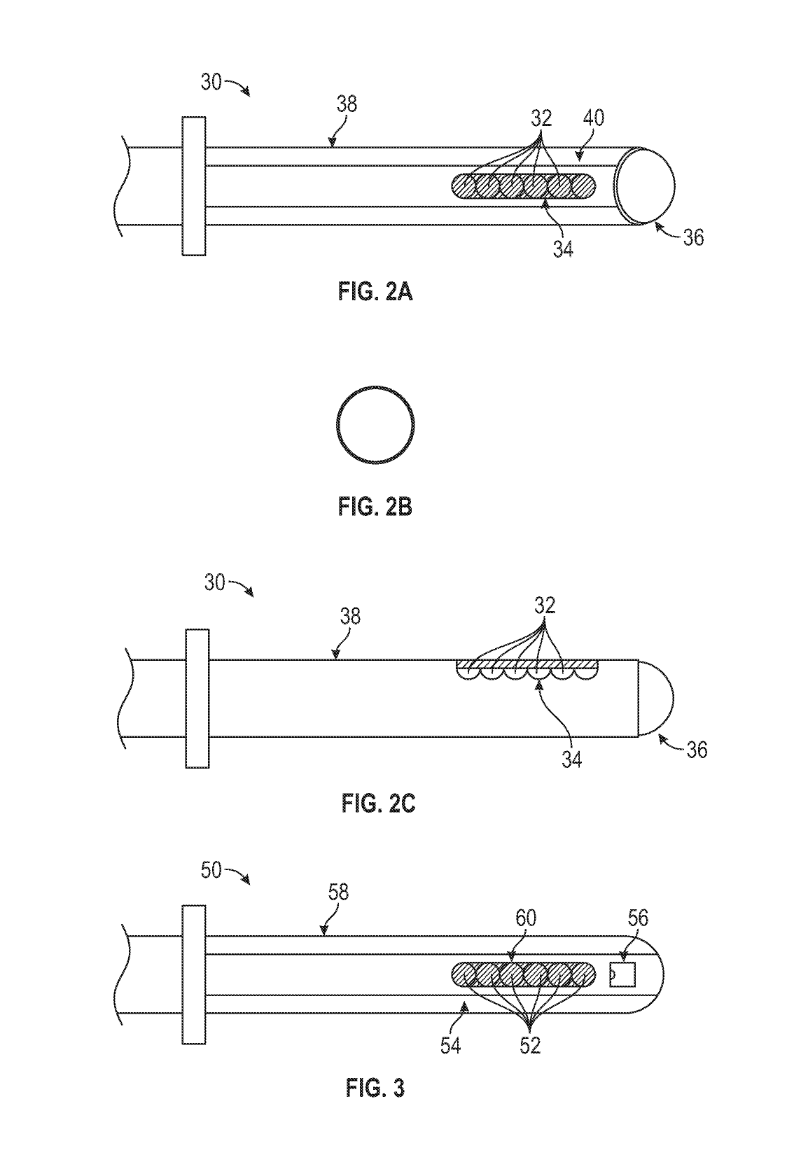

FIGS. 2A-2C show an applicator 30 according to another embodiment, with one or more transducers 32 and/or a transducer array 34 as described with relation to FIG. 1. The applicator 30 may be the same or similar as the applicator 10 except that the applicator 30 may include a rollerball tip 36. That is, the applicator 30 may include one or more transducers 32, a transducer array 34, a body 38, and/or a treatment window 40 similar or the same as the corresponding parts of applicator 10. The rollerball tip 36 may be substantially spherical. The rollerball tip 36 may be a moveable member that may move with respect to the body 38 of the applicator 30. The rollerball tip 36 may rotate respective to the body 38 of the applicator 30. For example, the rollerball tip 36 may rotate within a groove or depression in the distal end of the body 38. The rollerball tip 36 may rotate with respect to the body 38 as the applicator 30 is moved along a surface, for example, along the tissue, target tissue, and/or interior of the vagina. As the rollerball tip 36 rotates along the surface, the rotational and/or longitudinal position changes along the surface may be sensed and communicated to the operator. The treatment window 40, including the one or more transducers 32 may be actuated automatically in response to sensed position changes.

FIG. 3 shows an applicator 50 according to another embodiment, with one or more transducers 52 and/or a transducer array 54 as described with relation to FIG. 1. The applicator 50 may be the same or similar as the applicator 10 except that the applicator 50 may include an optical window 56. That is, the applicator 50 may include one or more transducers 52, a transducer array 54, a body 58, and/or a treatment window 60 similar or the same as the corresponding parts of applicator 10.

With continued reference to FIGS. 2A-2C and 3, the rollerball tip 36 and/or the optical window 56 may be an encoder for sensing rotational and/or longitudinal position changes of the applicators 30, 50, respectively. The encoder may be a rotary encoder, linear encoder, or combinations thereof. The encoder, rollerball tip 36, and optical window 56 may be electronically coupled to the applicators 30, 50 and/or to the workstation with necessary wires and circuits. The encoder may convert the rotary position of the applicators 30, 50 to an electronic signal. The encoder may convert the linear position of the applicators 30, 50 to an electronic signal. The electronic signal may include information regarding the rotary position, linear position, or both. The electronic signal may be communicated to the workstation, applicator, a computer, and/or the operator. The electronic signal may represent the rotary and/or linear position of the applicator 30, 50 within the vagina. The electronic signal may be used to map the vagina or automatically operate the applicators 30, 50 to delivery one or more treatment energies. The encoder may sense the motion of the applicator 30, 50 as the applicator 30, 50 and/or rollerball tip 36 rotates, moves laterally or longitudinally, or is otherwise manipulated and moved, within the vagina. The encoder may sense the motion of the applicator 30, 50 and/or rollerball tip 36 as the applicator 30, 50 is moved along the long axis either into or out of the vagina.

The operation of the applicator 30, 50 may be the same as the applicator 10 previously described. The treatment window 40, 60 may be aligned with and/or placed on the target tissue and activated in the same manner previously described with relation to FIG. 1. The applicator 30, 50 may be moved in the vagina to a subsequent location as previously described with relation to FIG. 1. Repeated pulses of energy (such as from the TUS) may be activated automatically as preselected rotational and/or longitudinal motion is sensed by the encoder as the applicator 30, 50 or portions of the applicator 30, 50 are moved over the target tissue. Repeated pulses of energy may be provided at the first target tissue location and/or subsequent target tissue locations. FIGS. 2A-2C and 3 may also be provided with Ultrasound Imaging (USI) as described with relation to the transducer of FIG. 1 to assist in location of the applicator within the vagina. FIGS. 2A-2C and 3 may be combined such that an applicator as described with relation to FIG. 1 is provided with both a rollerball tip 36 and an optical window 56.

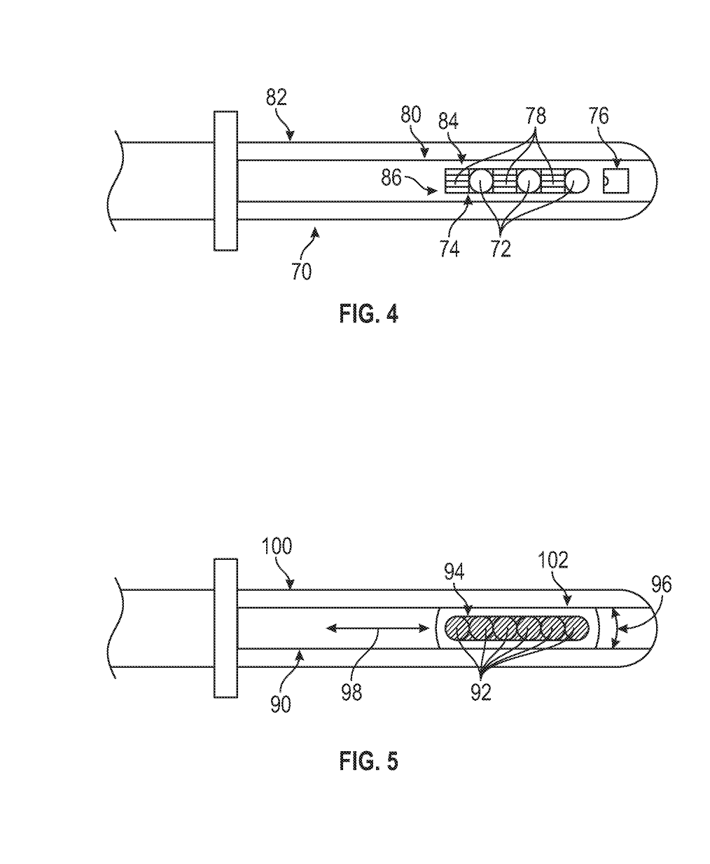

FIG. 4 shows an applicator 70 according to another embodiment, with one or more transducers 72 and/or a transducer array 74 as described with relation to FIG. 1. The applicator 70 may include one or more transducers 72, a transducer array 74, a body 82, and/or a treatment window 80 similar or the same as the corresponding parts of applicator 10. The applicator 70 may be the same or similar as the applicator 10 except that the applicator 70 may include an optical window 76 and/or one or more electrodes 78 in the treatment window 80. The one or more electrodes 78 may be arranged in an array 84, such as a linear array.

The applicator 70 of FIG. 4 may include one or more electrodes 78 in the treatment window 80, which may be the same or similar as the treatment window 18 described in FIG. 1. The one or more electrodes 78 may provide Monopolar Radiofrequency (MRF), Microwave Radiofrequency (MWRF), and/or Alternating Magnetic Field (AMF) treatment energies. Although the previously mentioned energies are different forms of energy, all are capable of producing therapeutic temperature increases in the target tissue. Monopolar radiofrequency (MRF) and microwave radio frequency (MWRF) differ primarily in their frequency and polarity configuration. The MRF uses a return pad to complete the circuit that delivers radiofrequency current, while the MWRF is a unipolar arrangement where the result is an electromagnetic field disruption that does not deliver current at all. Alternating Magnetic Field (AMF) is similar to MWRF in that there is no current delivered and additionally, both create electromagnetic field disruptions that cause repeated re-orientation of the hydrogen molecules in water and other tissue constituents. The one or more electrodes 78 may be included in addition to the one or more transducers 72 and/or the transducer array 74, or may replace some or all of the one or more transducers 72 in the transducer array 74. Any combination of one or more transducers 72 (such as TUS) and one or more electrodes 78 (such as MRF, MWRF, and AMF) may be provided in an array 86 for treatment of a target tissue. One or more of MRF, MWRF, and AMF may be provided in the applicator 70.

The applicator 70 of FIG. 4 is depicted with an optical window 76 which may operate in the same or similar manner as described for the optical window 56 in FIG. 3. The applicator 70 of FIG. 4 may be provided with a rollerball tip 36 (FIG. 2) in place of or in addition to the optical window 76. Alternatively, the optical window 76 and/or rollerball tip 36 may be omitted. Alternatively, USI may be included, as previously described, for locating the applicator 70 within the target. The applicator 70 of FIG. 4 may be operated in the same manner as described with relation to any of FIGS. 1-3. The treatment window 80 may be aligned with and/or placed on the target tissue and activated in the same manner previously described with relation to any of FIGS. 1-3. The applicator 70 may be moved in the vagina as previously described with relation to any of FIGS. 1-3. Repeated pulses of energy (such as from the one or more transducers 72 and/or the one or more electrodes 78) may be activated automatically as preselected rotational and/or longitudinal motion is sensed by the encoder (e.g. encoder or optical window 76) as the applicator 70 is moved over the target tissue as described previously in FIGS. 2 and 3. The applicator 70 of FIG. 4 may also be provided with Ultrasound Imaging (USI) as described with relation to the applicator 10 of FIG. 1 to assist in location of the applicator 70 within the vagina. The applicator 70 may be positioned by direct observation, inferred relative to anatomical landmarks, positioned with the assistance of USI, or positioned with the assistance of USI and a rollerball tip and/or optical window.

FIG. 5 is directed to an applicator with rotation and translation capabilities. The applicator may allow for the treatment window to move rotationally and translate longitudinally along the applicator to allow more precise and targeted control of the treatment window with respect to the target tissue. The treatment window may move with respect to the applicator body such that the applicator body may be held stationary while the treatment window translates or rotates along or around the applicator. These capabilities may be automated by a moveable member, for example with a motor and/or a moveable sleeve to which the treatment window is coupled, although other moveable members may be provided. An operator may map or scout an area (e.g. the vagina) with any of the aforementioned locating techniques. The operator may then insert the applicator into the area and move the treatment window, preferably automatically, to align with a target tissue or a target treatment area. The treatment window may deliver the treatment energy and then automatically move to the next location along/around the applicator to deliver the next treatment energy to the next target tissue.

FIG. 5 shows in one implementation of an applicator 90 according to another embodiment, with one or more transducers 92 and/or a transducer array 102 similar or the same as described with relation to FIG. 1. The applicator 90 may include one or more transducers 92, a transducer array 102, a body 100, and/or a treatment window 94 similar to or the same as the corresponding parts of applicator 10 of FIG. 1. The applicator 90 may be the same or similar as the applicator 10 except that the applicator 90 may include a motorized portion. The motorized portion may be a moveable member moveable with respect to the body 100 of the applicator 90. The moveable member may allow the treatment window 94 to move with respect to the body 100 of the applicator 90. The treatment window 94 may be moved at any increment of 360 degrees around the body 100 of the applicator 90. The treatment window 94 may be moved clockwise or counter clockwise. The treatment window 94 may be moved any increment along the length (e.g. along the 10 cm length) of the applicator 90.

The motorized portion of applicator 90 may include a motor (not visible). The motorized portion may be programmed to move the treatment window 94 over a prescribed rotational position along arrows 96 and/or longitudinal position along arrows 98 along the body 100. The treatment window 94 may be moved longitudinally along the exterior surface of the body 100 toward and away from a distal tip of the body 100. The treatment window 94 may be moved rotationally to other sides (e.g. a side located 90 degrees or 180 degrees around the circumference of the body 100) of the applicator 90. The treatment window 94 may be coupled to a cylindrical sleeve (not visible) coupled to an outer surface of the body 100. The cylindrical sleeve may be coupled in a track, cam, or other connection type which allows relative movement between the cylindrical sleeve (i.e. the treatment window 94) and the body 100. In this manner, the applicator 90 may be inserted into the vagina or near a treatment area. Once placed in the desired location, the applicator 90 may remain stationary and the treatment window 94 may be moved relative to the applicator 90 to align the treatment window 94 with one or more target tissues for delivery of the treatment energies. More than one treatment window 94 may be provided. One or more of the treatment windows 94 may be moveable in the aforementioned manner.

The applicator 90 may allow automated rotation and/or translation of the treatment window 94. The treatment window 94 may be placed at a predetermined “zero point” or beginning position. When activated, the treatment window 94 may automatically rotate in the direction of arrows 96 (clockwise and/or counterclockwise) and/or move longitudinally in the direction of arrows 98 (to and fro) with respect to the body 100 of the applicator 90. The treatment window 94 may rotate up to 360 degrees around the exterior surface of the body 100 of the applicator 90. The treatment window 94 may move longitudinally up to the entire length of the body 100 of the applicator 90.

The movement of the treatment window 94 around and/or along the applicator 90 may allow for an image of the vaginal wall and/or tissues to be generated. The operator may select areas of the generated image to be target tissue areas of the treatment. The operator may select areas of the generated image to be excluded from the treatment and/or may select areas of the generated image to be the target tissue for treatment.

With continued reference to FIG. 5, delivery of the treatment energy to the target tissue areas may be activated through sustained closure of the activation device (not depicted). The tissue to be targeted with the treatment energy may be selected based on location and/or functional nature. Once the target tissue is selected, the specific location of the target tissue may be determined by one of the previously mentioned techniques, including, for example, location relative to anatomical landmarks, direct observation, or imaging by ultrasound, or combinations thereof. The treatment window 94 may be moved (e.g. in the direction of arrows 96 and/or 98) before or during treatment to align and position on the target tissue. After delivery of the treatment energies to the selected target tissues, the treatment window 94 may be returned to its beginning or “zero point” location. The applicator 90 may be moved to a subsequent location in the vagina or removed from the vagina if treatment is complete. The treatment window 94 may be activated at the subsequent location to rotate and/or move longitudinally (e.g. in the direction of arrows 96 and 98, respectively), as previously described, to map the subsequent location of the vagina wall and tissues. A map may be generated and the operator may again select areas to include or exclude from the treatment. The treatment energies may be delivered to the target tissues through sustained closure of the activation device. The process may be repeated as needed at any number of vaginal locations until the full treatment is provided. Alternatively, the process may be completed after the first placement. After the delivery of the final treatment energy, the applicator 90 may be removed from the vagina.

The applicator 90 of FIG. 5 may also be provided with Ultrasound Imaging (USI) as described with relation to the applicator 10 of FIG. 1 to assist in location of the applicator 90 within the vagina. The applicator 90 may be positioned by direct observation, inferred relative to anatomical landmarks, positioned with the assistance of USI, or positioned with the assistance of USI and a rollerball tip and/or optical window. The applicator 90 of FIG. 5 may be provided with the rollerball tip 36 (FIG. 2A-2C) and/or the optical window 56 (FIG. 3). The applicator 90 of FIG. 5 may be provided with one or more electrodes 78 (FIG. 4).

The applicator 10, 30, 50, 70, 90 of any or all of FIGS. 1-5 may be any suitable length for performing the treatment in the vagina. For example, the length of the applicator may be any length up to about 10 cm. The length may be 9 cm-11 cm, 8 cm-12 cm, 10 cm-14 cm, 9 cm-13 cm, etc. Although the applicators of FIGS. 1-5 are described as being a cylindrical or tubular shape, other shapes may be employed for performing the treatment. Additionally, although the treatment window is depicted on one side of the applicator, the treatment window may be placed such that it covers multiple sides, alternating sides, etc. Alternatively, more than one transducer/electrode array and/or treatment window may be provided on the same or different sides for performing the treatment. The treatment window may be size and shaped to allow for positioning of the transducer over the desired target tissue and/or more than one target tissue.

Although the foregoing description is described with use in a vagina for treatment of Stress Urinary Incontinence in women, the treatment may be used to treat Stress Urinary Incontinence in men. In such instances, the shape and size of the applicator may be altered to be suitable for use in men. Additionally, the treatment may treat other conditions and is not limited to treatment of Stress Urinary Incontinence. Additionally, any of the treatment energies described may be applied in an alternating or predetermined sequence. For example, where one or more types of transducers and/or one or more types of electrodes are provide, the application of the treatment energies from the respective one or more transducers and/or one or more electrodes may be performed in an alternating, sequenced, or otherwise predetermined manner.

According to embodiments of the invention, a treatment of Stress Urinary Incontinence is provided. The treatment may comprise treating a target tissue in the vagina to support the bladder neck and/or urethra as it descends into the vaginal space. The treatment may change the target tissue by strengthening and thickening the tissue in the anterior vagina to provide the aforementioned support. The treatment energies may strengthen and thicken the tissue. The controlled delivery of monopolar radiofrequency energy (or other treatment energy) to the target tissue of the vaginal introitus may increase temperature and activate fibroblast activity in the treated area. This may result in connective tissue remodeling and augmentation which may increase strength and thickness of the vaginal introitus. These same reactions may be produced through the administration of alternative energies also capable of elevating the temperature of the target tissue to therapeutic levels. Selectively treating the area of the urethral prominence in the anterior vagina with heat inducing energy may cause connective tissue remodeling that may reduce the incidence and severity of stress urinary incontinence by enabling the anterior vaginal tissue to more competently impede the free descent of the urethra into vaginal space where it leaks urine when a patient coughs, laughs, sneezes or experiences other activity causing a sharp rise in intra-abdominal pressures. By opposing the descent of the urethra, the tissue may be made to flatten itself along a greater length, increasing the patency of its sphincter function. The treatment may provide substantially complete relief from Stress Urinary Incontinence related events and may be durable. The effects of the treatment may last between about six months and about 12 months, for example. Thus, novel devices, systems, and methods are provided herein for strengthening and thickening the tissue in the vagina. The target tissues may be of the mucosa, lamina propria, and/or fibromuscular layers of the vagina. The target tissue may be other tissues interior to or exterior to the vagina.

The applicator of the present disclosure may allow for controlled application of treatments to a tissue without the operator's ability to visualize the tissue (e.g. the operator may be “blind” to the treatment location). The rollerball tip and/or optical window may allow for the operator to direct the applicator around tissues and surfaces not visible. Furthermore, the rollerball tip and/or optical window may allow for more precise and/or finite control of the applicator. For example, the rollerball tip may be able to sense small or minute changes in rotational and/or longitudinal movement of the applicator. This may allow for more precise control and/or more precise target areas for treatment. The applicator of the present disclosure may preferably delivery non-damaging, non-ablative treatment energies. The applicator of the present disclosure may cause no tissue damage. The applicator of the present disclosure may remodel tissue.

The invention of the present disclosure is a multiple energy based workstation consisting of a device console with power supply, display, a set of one or more delivery systems and control electronics to provide visualization of the target tissue and to select, scale and activate treatment parameters for the delivery of Therapeutic Ultrasound (TUS), Monopolar Radiofrequency (MRF), Microwave Radiofrequency (MWRF) or Alternating Magnetic Field (AMF) treatment energies, individually or in combination.

In one embodiment, an applicator with a length of up to 10 cm and a tubular shape appropriate for use in the vagina is attached to the system. The applicator has a side-facing TUS array along one side, which is placed on the target tissue either by direct observation or inferentially relative to anatomical landmarks. A single TUS energy activation occurs by closure and release of a foot switch or handpiece trigger. After the delivery of TUS energy, the applicator is repositioned, again either by direct observation or inferentially relative to anatomical landmarks and additional TUS emissions are activated as required to treat the complete target area. The size and shape of the active portion of the applicator and the implementation of a linear TUS array are important in that they enable access to and positioning of the TUS array over the desired target tissue. The foot switch and/or handpiece trigger is important as it controls the emission of TUS energy only as desired by the treating clinician.

In another embodiment, the applicator as described previously combines TUS with Ultrasound Imaging (USI). The side of the applicator corresponding to the location of the combined TUS/USI array is placed on the target tissue using ultrasonic imaging of relevant anatomical landmarks. A single TUS energy activation occurs by closure and release of a foot switch or handpiece trigger. After the delivery of TUS energy, the applicator is repositioned with the aid of USI, and additional TUS emissions are activated as required to treat the complete target area. The size and shape of the active portion of the applicator and the implementation of a linear TUS/USI array are important in that they enable access to and positioning of the combined TUS/USI array over the desired target tissue. The footswitch and/or handpiece trigger is important as it controls the emission of TUS energy only as desired by the treating clinician.

In another embodiment, the applicator described above, combining TUS with Ultrasound Imaging (USI), also has a rollerball tip encoder or optical window to sense rotational and longitudinal position changes. The side of the applicator corresponding to the location of the combined TUS/USI array is placed on the target tissue using ultrasonic imaging of relevant anatomical landmarks. A single TUS energy activation occurs by closure and release of a foot switch or handpiece trigger. Repeated pulses of TUS energy may be activated automatically as preselected rotational and/or longitudinal motion is sensed by the encoder as the tip moves over the target tissue. The size and shape of the active portion of the applicator and the implementation of a linear TUS/USI array are important in that they enable access to and positioning of the combined TUS/USI array over the desired target tissue. The implementation of the rollerball encoder tip or optical window is important as the sensing of rotational and/or longitudinal translation allows controlled fractional coverage of the tissue target with treatment energy. Releasing the foot switch or handpiece trigger stops the delivery of treatment energy regardless of other sensors.

In another embodiment, the applicator described above, combining TUS with Ultrasound Imaging (USI) also has a motorized, portion that may be programmed to move the treatment array over a prescribed rotational and/or longitudinal distance. The side of the applicator corresponding to the location of the combined TUS/USI array could be placed at a pre-determined “zero point.” When triggered to do so, the TUS/USI transducer could automatically rotate up to 360 degrees (either clockwise or counter clockwise), or move linearly over the 10 cm length of the applicator (and incrementally along the 10 cm length, e.g. move 1 cm in either longitudinal direction, or 2 cm, etc.), generating a scout image of the vaginal wall and underlying tissues. The operator may interact with the system to select the parts of the image that correspond to intended targets, and exclude areas that are not targets. An automated delivery of treatment energy to the intended targets may be activated by sustained closure of a foot switch or handpiece trigger. At the end of the sweep, the rotating portion of the applicator would return to a zero position and await repositioning for the next treatment sweep. The size and shape of the active portion of the applicator and the implementation of a linear TUS/USI array are important in that they enable access to and positioning of the combined TUS/USI array over the desired target tissue. The automated rotational and/or longitudinal translation of the TUS/USI array is important in that it controls where the therapeutic energy is delivered and allows precise control of the fractional density of the coverage on the target tissue. The foot switch or handpiece triggers remain important, operating in a deadman fashion where closure initiates the delivery of treatment energy and release would halt the delivery of treatment energy regardless of the status or position of the various, sensors or transducers in the applicator.

Other embodiments may substitute one or more combinations of Monopolar Radiofrequency (MRF), Microwave Radiofrequency (MWRF) or Alternating Magnetic Field (AMF) treatment energies into each of the scenarios detailed above, in place of or in addition to TUS. These alternate or combination transducer arrays may be positioned by direct observation or inferred relative to anatomical landmarks, positioned with the assistance of USI, or with the assistance of USI and automated rotational or longitudinal translation to create a controlled fractional coverage of the intended treatment surface over target tissue.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A system for delivery of therapeutic energies to a target tissue, the system comprising:
an applicator having a body;
a treatment window located on the body of the applicator; and
a moveable member coupled to the applicator, the moveable member configured to move with respect to the body,
wherein the moveable member is configured to move the treatment window into alignment with the target tissue for delivery of a treatment energy.

2. The system of claim 1, wherein the moveable member is a rollerball tip coupled to a distal end of the body.

3. The system of claim 2, wherein the rollerball tip includes an encoder configured to sense a rotational and longitudinal position of the applicator.

4. The system of claim 3, wherein the treatment window is configured to deliver the treatment energy automatically based on the sensed position.

5. The system of claim 3, wherein the treatment window is configured to delivery repeated pulses automatically based on a preselected rotational or longitudinal position sensed by the encoder.

6. The system of claim 2, wherein the rollerball tip rotates relative to the body.

7. The system of claim 1, wherein the moveable member is a motorized portion coupling the treatment window to the applicator.

8. The system of claim 7, wherein the motorized portion is configured to move the treatment window rotational and longitudinally along the body of the applicator.

9. The system of claim 7, wherein the motorized portion is configured to align the treatment window with the target tissue for delivery of the treatment energy.

10. The system of claim 1, wherein the applicator is configured to scout an area to determine one or more target tissues, and wherein the moveable member moves the treatment window from a zero position to a treatment position aligned with one of the one or more target tissues to deliver the treatment energy to the one of the one or more target tissues.

11. The system of claim 10, wherein the moveable member moves the treatment window from the treatment position to the zero position after delivery of the treatment energy.

12. The system of claim 11, wherein the moveable member moves the treatment window automatically.

13. A system for endovaginal delivery of therapeutic energies, the system comprising:
an applicator comprising:
a body; and
a treatment window located on a side of the body, the treatment window comprising one or more transducers,
wherein the applicator is configured to be positioned within a vagina of a patient and,
wherein the treatment window is configured to be aligned with a target tissue in the vagina and the one or more transducers are configured to deliver a treatment energy to the target tissue.

14. The system of claim 13, further comprising an activation device configured to turn the one or more transducers on and off.

15. The system of claim 13, wherein the treatment energy is one or more of Therapeutic Ultrasound (TUS), Monopolar Radiofrequency (MRF), Microwave Radiofrequency (MWRF), Alternating Magnetic Field (AMF), or combinations thereof.

16. The system of claim 13, wherein the body of the applicator comprises a substantially cylindrical portion and a rounded distal tip and wherein the treatment window is located on the substantially cylindrical portion.

17. The system of claim 16, wherein the treatment window creates a substantially flat portion on the substantially cylindrical portion of the body.

18. The system of claim 13, wherein the treatment window is moveable longitudinally along the body and rotationally around the body and wherein the treatment window is configured to map an area of the vagina for selecting one or more target tissues.

19. The system of claim 13, further wherein the one or more transducers form a transducer array.

20. The system of claim 19, wherein the transducer array is a therapeutic ultrasound array or linear transducer array.

21. The system of claim 13, further comprising one or more electrodes.

22. The system of claim 21, wherein the one or more electrodes form an electrode array.

23. The system of claim 22, wherein the one or more transducers and the one or more electrodes deliver one or more treatment energies including Therapeutic Ultrasound (TUS), Monopolar Radiofrequency (MRF), Microwave Radiofrequency (MWRF), Alternating Magnetic Field (AMF), or combinations thereof.

24. The system of claim 13, further comprising one or more of ultrasound imaging, a rollerball tip, or an optical window on the applicator.

25. The system of claim 24, wherein the rollerball tip and optical window include an encoder for sensing rotational and longitudinal position changes of the applicator.

26. The system of claim 13, wherein the applicator is located with ultrasound imaging and the treatment window delivers a therapeutic ultrasound treatment energy.

27. A method for treating a target tissue with therapeutic energies, the method comprising:
inserting an applicator with a treatment window into a vagina of a patient;
locating the treatment window adjacent a target tissue of the vagina;
activating one or more transducers in the treatment window;
delivering a treatment energy to the target tissue with the one or more transducers;
relocating the applicator to a second target tissue in the vagina without removing the applicator from the vagina; and
delivering the treatment energy to the second target tissue.

28. The method of claim 27, further comprising locating the applicator within the vagina with one or more of direct observation, inference relative to anatomical landmarks on an anterior vulva, or ultrasonic imaging.

29. The method of claim 28, wherein direct observation includes observation of the length and location of the urethral prominence.

30. The method of claim 27, wherein the target tissue is one or more of mucosa, lamina propria, or fibromuscular layers.

31. The method of claim 27, further comprising sensing rotational and longitudinal position changes of the applicator and automatically activating the one or more transducers based on the sensed position changes.

32. The method of claim 27, wherein activating one or more transducers includes operating an activation device.

33. The method of claim 27, further comprising automatically pulsing treatment energies when the applicator is located at at least one of the target tissue or the second target tissue.

34. The method of claim 27, further comprising mapping an area of the vagina by moving the treatment window longitudinally along and rotational around the applicator; and selecting one or more target tissues based on the area mapped.
